(19) United States
(12) Patent Application Publication
(10) Pub. No.: US 2009/0201466 A1
(43) Pub. Date: Aug. 13, 2009

(54) Goggles

(76) Inventors: Stephen M. Knecht, Miami, FL (US); Scott Chan, Hong Kong (HK); Leo Cheung, Hong Kong (HK)

Correspondence Address: WITHERS & KEYS, LLC, P. O. BOX 71355, MARIETTA, GA, 30007-1355, US

(21) Appl. No.: 11/911,194

(22) PCT Filed: Apr. 11, 2006
(86) PCT No.: PCT/US2006/013512

§ 371 Date: May 21, 2008

Related U.S. Application Data
(60) Provisional application No. 60/670,084, filed on Apr. 11, 2005.
(60) Provisional application No. 60/719,523, filed on Sep. 22, 2005.

(51) Int. Cl. A61B 3/14; A61F 9/02

(52) U.S. Cl. 351/206; 2/431

(57) ABSTRACT

Goggles provide various features including skirt that mounts to a frame of the goggles. The skirt may be elastic and may include damper portions at least partially extending into the elastic skirt to allow the elastic skirt to have travel and to thereby better conform to the facial features of a subject. The goggles may include an overhead mounting location such as a chamber that encloses movement, such as infrared light sources and/or cameras. Furthermore, the goggles may include infrared mirrors that are retained by a bracket mounted to the frame, where the bracket has a portion affixed to the frame that retains another portion that is movable and that is affixed to the infrared mirrors.

### RELATED APPLICATIONS

The present application claims priority to U.S. Provisional Application 60/670,084, filed on Apr. 11, 2005, and entitled BALANCE AND VESTIBULAR DISORDER DIAGNOSIS AND REHABILITATION, which is incorporated herein by reference. The present application also claims priority to U.S. Provisional Application 60/719,523, filed on Sep. 22, 2005, and entitled BALANCE AND VESTIBULAR DISORDER DIAGNOSIS AND REHABILITATION, which is also incorporated herein by reference.

### TECHNICAL FIELD

The present application is related to eyewear, namely goggles.

### BACKGROUND

Goggles are worn by subjects for many reasons. For example, the subject may be participating in an activity where the goggles provide necessary eye protection. As another example, the subject may be participating in a virtual reality activity where the goggles provide a visual display. As another example, the subject may be undergoing a test related to the eyes of the subject, such as a test to measure visual acuity or ocular movement, where the goggles provide cameras that capture video signals of the eyes of the subject.

In these instances and others, conventional goggles may have various shortcomings. Goggles generally have a skirt that contacts the face of the subject and the skirt may not adapt well to the facial features of the subject. As a result, the goggles may be uncomfortable, may not remain fixed in the proper position, and so forth. Furthermore, there may be gaps introduced where the skirt does not adequately contact the face of the subject such that the skirt fails to seal out ambient conditions. For example, where an activity is done in cold weather, a poor fit between the skirt and the face allows cold air to reach the eyes of the subject. As another example, where the interior of the goggles needs to be dark, such as when performing testing of a response of the eyes to darkness or where creating a virtual reality with video displays, a poor fit between the skirt and the face allows ambient light to enter which causes interference with the intended activity.

Conventional goggles used for ocular movement tests may have shortcomings directly related to the testing. For example, such conventional goggles contain one or more cameras that are typically mounted to the side of the eyes, which blocks at least a portion of the peripheral view of the patient. As another example, such conventional goggles typically utilize an infrared mirror where an infrared camera is focused on the mirror but the mirror is in a fixed position such that the image captured by the camera may not be ideal for all subjects.

### SUMMARY

Embodiments address these issues and others by providing goggles having various features. For example, according to an embodiment, the goggles have a skirt that has damper sections that provide travel such that the skirt is more likely to adapt to varying facial features of subjects. According to an embodiment, the goggles have an infrared light source mounted over and ahead of the portions where the eyes look through so that the infrared light source does not interfere with the peripheral view of the subject. According to an embodiment, the goggles have an infrared camera mounted over and ahead of the portions where the eyes look through so that the infrared camera does not interfere with the peripheral view of the subject. According to an embodiment, the goggles have infrared mirrors that are mounted via a bracket that provides for adjustment of the angle of the infrared mirrors.

One embodiment is a pair of goggles that includes an elastic skirt forming at least one aperture and having a curvature extending from a first side of the elastic skirt to a second side of the elastic skirt. The elastic skirt has a facial side, an outer rim, and a frame side, the outer rim defining a nose indentation, the outer rim including first, second and third damper portions partially extending into the elastic skirt. The first damper portion is formed along the curvature in a top portion of the outer rim opposite the nose indentation, and the second damper portion is formed along the curvature in a bottom portion of the outer rim between the first side and the nose indentation, and the third damper portion being formed between the second side and the nose indentation. A frame is attached to the frame side of the elastic skirt, the frame being more rigid than the elastic skirt, the frame having a curvature extending from a first side of the frame to a second side of the frame, the curvature of the frame coinciding with the curvature of the elastic skirt.

Another embodiment is a pair of goggles that includes a skirt forming at least one aperture, the skirt having a facial side, an outer rim, and a frame side. A frame is attached to the frame side of the skirt, the frame being more rigid than the skirt, wherein the skirt forms an aperture having two bays separated by a narrow portion formed by a nose indentation of the skirt and wherein the frame forms an aperture having two bays aligned with the two bays of the skirt and separated by a narrow portion formed by a nose indentation of the frame that is aligned with the nose indentation of the skirt. A first infrared mirror is coupled to the frame and is disposed beneath the chamber and in alignment with the first bay of the frame. A second infrared mirror is coupled to the frame and is disposed beneath the chamber and in alignment with the second bay of the frame. A first infrared light source is coupled to the frame such that the first infrared light sourced is over and ahead of the first bay of the frame, the first infrared light source being off-center relative to the first infrared mirror and being angled with respect to the frame so as to be aimed toward the center of the first infrared mirror. A second infrared light source is coupled to the frame such that the second infrared light source is over and ahead of the second bay of the frame, the second infrared light source being off-center relative to the second infrared mirror and being angled with respect to the frame so as to be aimed toward the center of the second infrared mirror.

Another embodiment is a pair of goggles that includes a skirt forming at least one aperture, the skirt having a facial side, an outer rim, and a frame side. A frame is attached to the frame side of the skirt, the frame being more rigid than the skirt, wherein the skirt forms an aperture having two bays separated by a narrow portion formed by a nose indentation of the skirt and wherein the frame forms an aperture having two bays aligned with the two bays of the skirt and separated by a narrow portion formed by a nose indentation of the frame that is aligned with the nose indentation of the skirt. A first infrared mirror is in alignment with the first bay of the frame, and a second infrared mirror in alignment with the second bay of the frame. A mirror bracket has a first portion affixed to the nose indentation of the frame and has a second portion affixed to the first and second infrared mirrors, wherein second portion is retained by and is movable relative to the first portion.

### DESCRIPTION OF THE DRAWINGS

FIG. 1A shows a front perspective view of one embodiment of goggles with an attached over cover as the goggles are worn by a subject.

FIG. 1B shows a front perspective view of one embodiment of goggles without a lower cover as the goggles are worn by a subject.

FIG. 2A shows lower front perspective view of one embodiment of goggles.

FIG. 2B shows a rear perspective view of one embodiment of goggles.

FIG. 3 shows a front view of one embodiment of goggles.

FIG. 4 shows a rear view of one embodiment of goggles.

FIG. 5 shows a side view of one embodiment of goggles.

FIG. 6 shows a top view of one embodiment of goggles.

FIG. 7 shows a bottom view of one embodiment of goggles.

FIG. 8 shows an exploded perspective view of one embodiment of goggles.

FIG. 9 shows a cross-sectional view of one embodiment of goggles.

FIG. 10 shows an exploded perspective view of a mirror adjustment assembly of one embodiment of goggles.

FIG. 11 shows a top view of a board of one embodiment of goggles.

FIG. 12 shows a cross-sectional view of the board.

FIG. 13 shows a front perspective view of one embodiment of a control box for use with various embodiments of the goggles.

FIG. 14 shows a rear perspective view of one embodiment of the control box for use with various embodiments of the goggles.

FIG. 15 shows an exploded perspective view of one embodiment of the control box for use with various embodiments of the goggles.

### DETAILED DESCRIPTION

Goggles are used to provide one of more various functions on behalf of a subject, such as eye protection, medical examination, and so forth. Various embodiments of goggles include a frame and a skirt. In certain embodiments, the skirt includes features to allow the skirt to better adapt to the face of the subject. In certain embodiments, the goggles include equipment for recording video and/or still image of ocular characteristics or movement. The equipment may include infrared light sources, infrared cameras, and infrared mirrors where the cameras are centered with the infrared mirrors while the infrared light sources are off-center but angled so as to be aimed at the center of the respective mirror. The equipment may further include a mirror adjustment bracket mounted to a nose indentation of the frame that allows the angle of the infrared mirror to be adjusted by manipulation of a portion of the bracket.

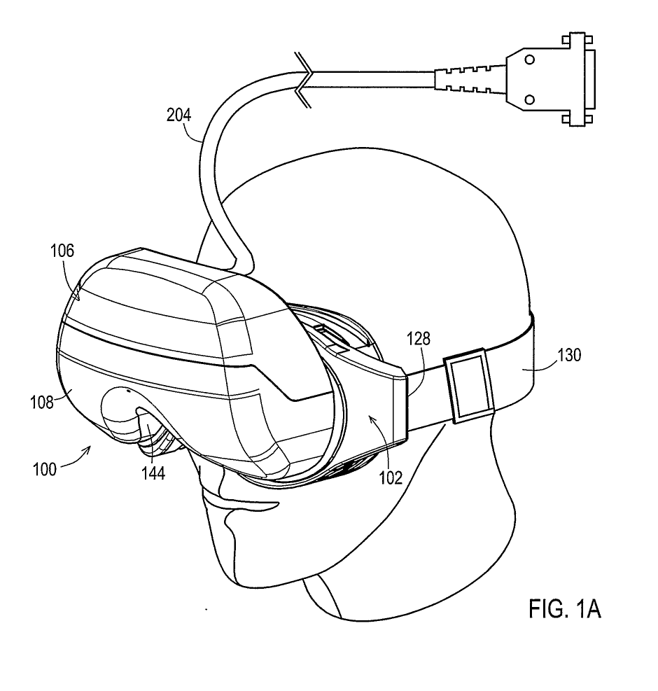

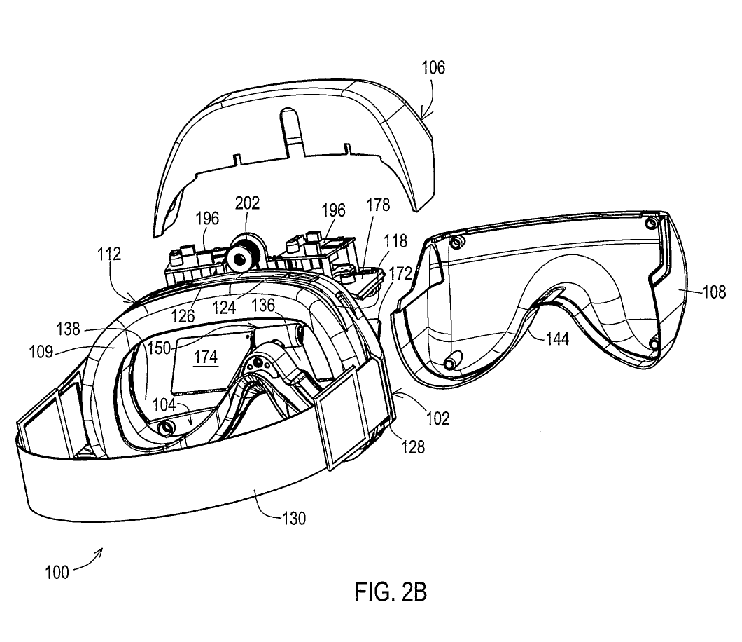

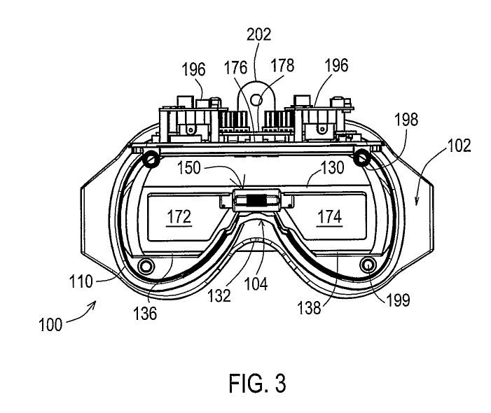

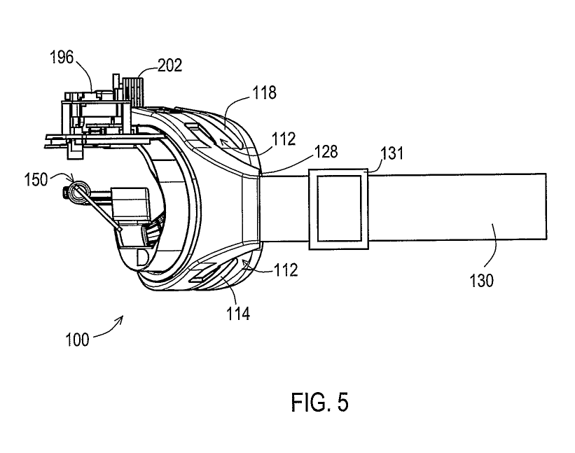

FIGS. 1A-7 show various views of one embodiment of goggles 100. This particular embodiment includes a skirt 102 mounted to a frame 104 and also includes equipment for capturing video footage of ocular movement. It will be appreciated that while the embodiment shown includes a particular skirt 102 and video capture equipment, other embodiments may provide for the skirt 102 without the video capture equipment or with different video capture configurations than that shown. It will also be appreciated that other embodiments may provide for the video capture equipment but with a different skirt than that shown.

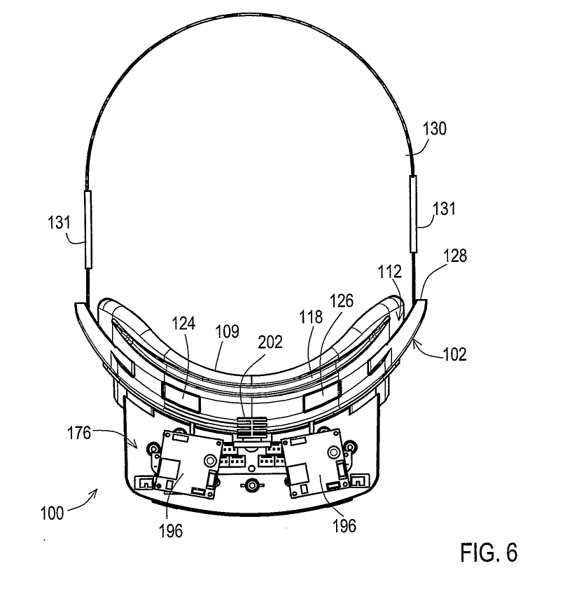

The pair of goggles 100 includes a skirt 102 that has a facial side 109 that contacts the face of the subject when the goggles 100 are worn. A frame 104 is attached to an outer side 110 of the skirt 102. In this example and as best seen in the exploded view of FIG. 8, the frame 104 has a ridge 105 about its periphery fits within a recess 107 of the skirt 102. Screws or other fasteners may be used to further attach the frame 104 to the skirt 102 in this example. Furthermore, as discussed below, a mirror bracket assembly mounted at a nose indentation 134 of the frame 104 also clamps the frame 104 to the skirt 102 at a nose indentation 132 of the skirt 102.

To hold the goggles 100 in place, a strap 130 may attach to the skirt 102. Attachments 128 on each side of the skirt 102 fixed the ends of the strap 130 to the goggles 100 and the strap 130 is then placed behind the head of the subject. The strap 130 has elasticity to enable the strap to be stretched to fit the subject and to maintain the position of the goggles 100. Additionally, buckles 131 may be provided to allow the strap 130 to be adjusted for varying head sizes.

To allow the goggles 100 to fit many different facial types and create an adequate seal for blocking out ambient conditions such as light and weather, the skirt 102 of this example has various characteristics. The skirt 102 may be made of an elastic material so that the skirt 102 has the ability to conform. One example of such an elastic material is a thermoplastic elastomer (TPE). In addition to utilizing an elastic material, the skirt 102 may be provided with dampers that allow the skirt to have travel. The dampers of this example are collapsible channels running along an outer rim 112 of the skirt 102 along the top from side to side and along the bottom from one side to the nose indentation 132.

The damper channel 118 runs along the top side and allows the skirt 102 to have travel and thereby conform to the forehead region of the face of the subject. A damper channel 116 runs along the bottom side, from a right side inward toward the nose indentation 132. Likewise, a damper channel 114 runs along the bottom side from a left side inward toward the nose indentation 132. The damper channels 114, 116 allow the skirt 102 to have travel and thereby conform to the upper cheek region of the face of the subject.

In addition to these damper channels, additional dampers may be provided such as the cut-outs 120, 122 along the bottom side and the cut-outs 124, 126 along the top side. The channels provide additional travel and contouring to the facial features of the subject.

The frame 104 is a more rigid material than the skirt 102 of this example. The frame 104 may be injection molded plastic, acrylonitrile butadiene styrene (ABS), or other similar material capable of retaining its shape. As can be seen, the frame 104 and the skirt 102 both have a curvature extending from one side to the next so that the curvature of the frame 104 is coincident with the curvature of the skirt 102 when the two are attached. This curvature aids in conforming to the face of the subject.

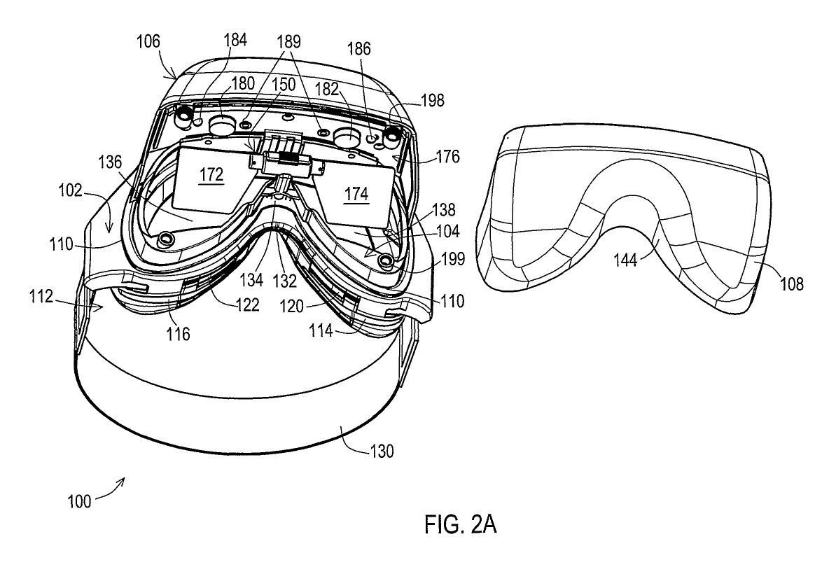

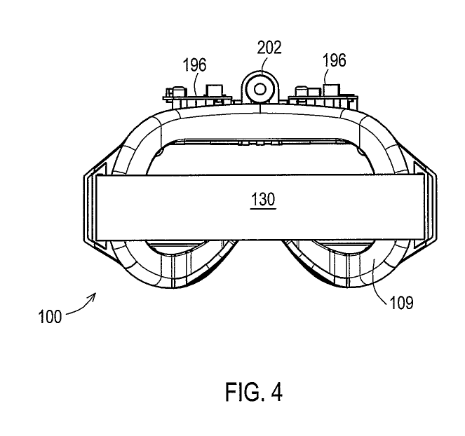

In addition to the frame 104 and skirt 102, this example provides an overhead chamber defined by a cabinet 106. This cabinet 106 and the chamber therein holds the video capture equipment of this embodiment. As seen in FIGS. 3 and 6 as well as in the exploded view of FIG. 8, the chamber includes a base section 176 having mounting posts 190 and apertures 192 where the mounting posts attach to the cabinet 106.

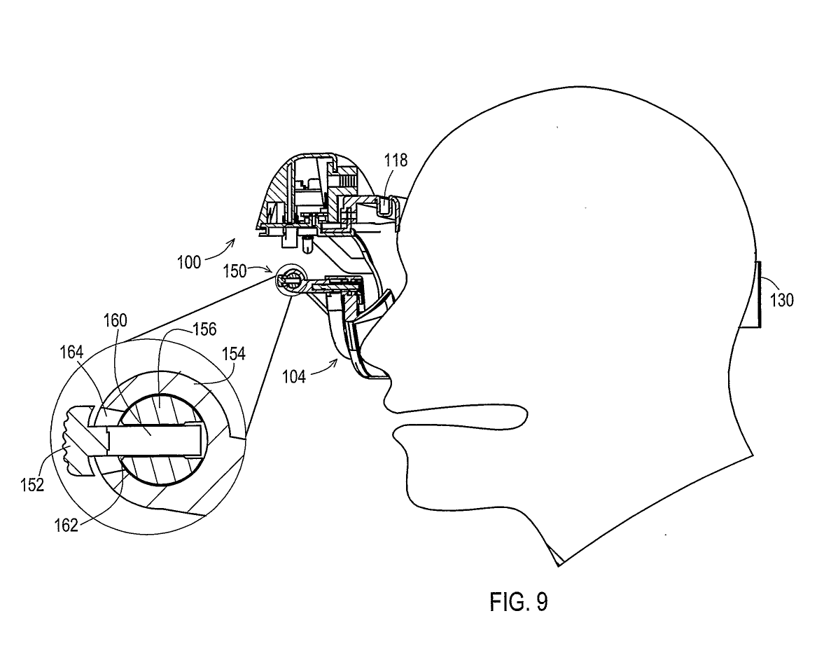

A circuit board 178, which rests upon the base section 176 and is shown in FIGS. 3, 6, 8, 11, and 12, includes recesses 188. Infrared camera assemblies 180 and 182 including circuit boards 196 then mount upon the circuit board 178 with the cameras 180, 182 extending through the recesses 188 and the apertures 192. Fixation and infrared light sources may be mounted to the circuit board to receive activation signals, as discussed below. The circuit boards 196 may also be interconnected to the circuit board 178 for sending and receiving signals via a main output connection from the circuit board 178 to external devices, such as a control box discussed below.

The infrared cameras 180, 182 are aimed downward so as to be focused on lenses 172, 174, respectively. These lenses 172, 174 have a coating that is reflective to infrared wavelengths, but generally transparent to visible wavelengths. Accordingly, the infrared cameras 180, 182 capture infrared images that appear on the infrared mirrors 172, 174. These infrared images are the infrared reflections of the eyes and surround facial areas of the subject wearing the goggles 100. The subject is able to see through the infrared mirror 172, 174 as they are transparent to visible wavelengths.

The cameras 172, 174 may be of various types. As one example, the cameras may be cameras that take infrared light and produce a National Television Standard Committee (NTSC) output signal. An example of an infrared camera producing an NTSC signal is the model MTV-03X10EH by Fujita of Japan.

In addition to the cameras, the cabinet 106 may house emission sources, including fixation sources and infrared sources. The fixation sources may be visible wavelength light emitting diodes (LEDs) 189 which may be used to adjust the amount of visible light seen by the subject when the cover 108 is in place. The presence of visible light while the cover 108 is in place may allow for observation of various medical conditions of the subject. The infrared sources may be infrared wavelength LEDs 184, 186 that cast infrared light upon the infrared mirrors 172, 174 which then direct the infrared light toward the eyes of the subject in order for it to be reflected back to the infrared mirrors 172, 174.

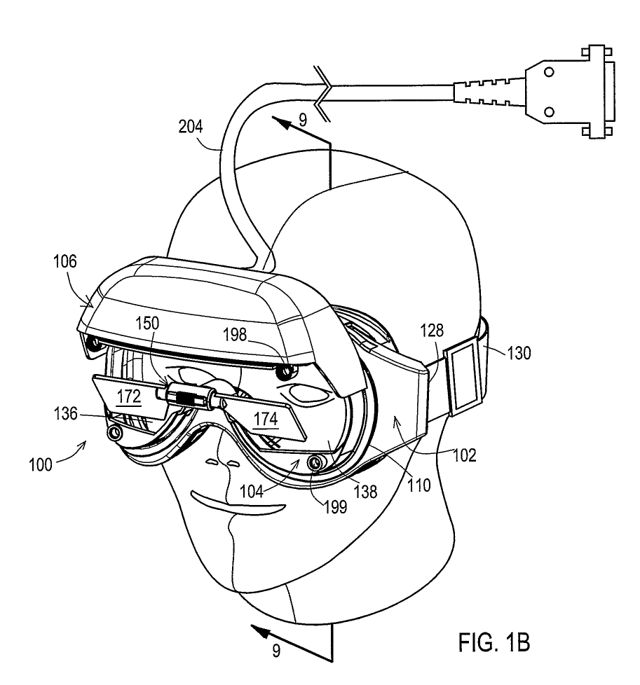

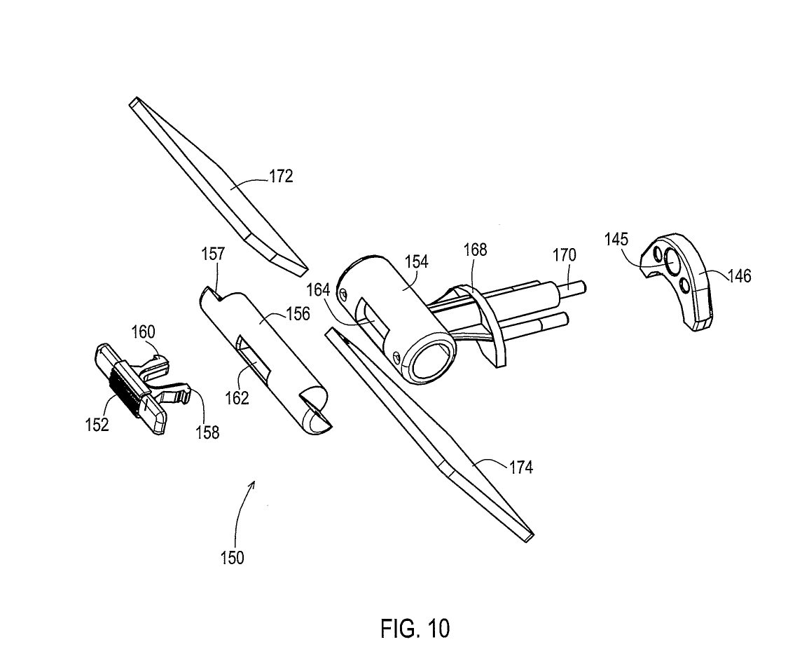

As shown in FIG. 2A, the cameras 180, 182 are positioned over the infrared mirrors 172, 174 and are aligned to be close to the centers of the mirrors 172, 174. To avoid the infrared LEDs 184, 186 from being in the field of view, these LEDs 184, 186 are placed adjacent to the cameras on the base 176. To account for the LEDs 184, 186 being placed away from the center of the mirrors 172, 174, the LEDs 184, 186 are mounted upon angled bases 194, as shown in FIG. 12 so that the LEDs 184, 186 are angled relative to the circuit board 178, mounting base 176, and frame 104. The angle is chosen in this example so that the LEDs 184, 186 are aimed at substantially the center of the infrared mirrors 172, 174 which then makes the most efficient use of the LEDs 184, 186 and allows only a single infrared LED to be used per eye. As shown, the chosen angle is 15 degrees. As also shown, the LEDs 184, 186 are spaced away from the cameras 180, 182 so that for the dispersion angle of the LEDs, about 60 degrees in this example, the cameras 180, 182 do not block the infrared illumination.

The skirt 102 and the frame 104 each have nose indentations 132, 134, respectively, in order to fit to the nose of the subject. The skirt 102 and the frame 104 each create an aperture through which the subject can see. The nose indentations 132, 134 create bays of the aperture, with nose indentation 132 of skirt 102 defining bays 140 and 142 and with the nose indentation 134 of frame 104 defining bays 136 and 138. The infrared mirrors 172, 174 are aligned with these bays so that the user sees through the bays and through the infrared mirrors 172, 174. As can be seen, the cameras 180, 182, and the LEDs 184, 186, and 189 are over and ahead of the bays 132, 134.

The infrared mirrors 172, 174 are coupled to the frame 104 via a nose piece mounting bracket assembly 150. This nose piece mounting bracket assembly 150 may be made of injection molded plastic or similar materials. In this particular example, the nose piece mounting bracket assembly 150 is constructed of a substantially transparent plastic so that it is less of a visible distraction to the subject.

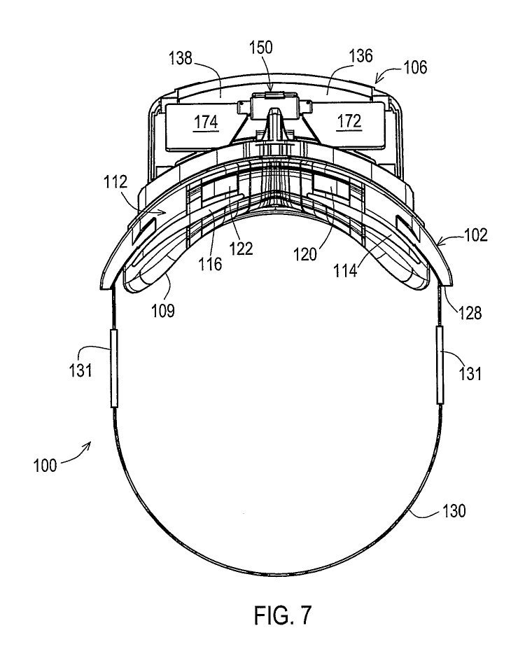

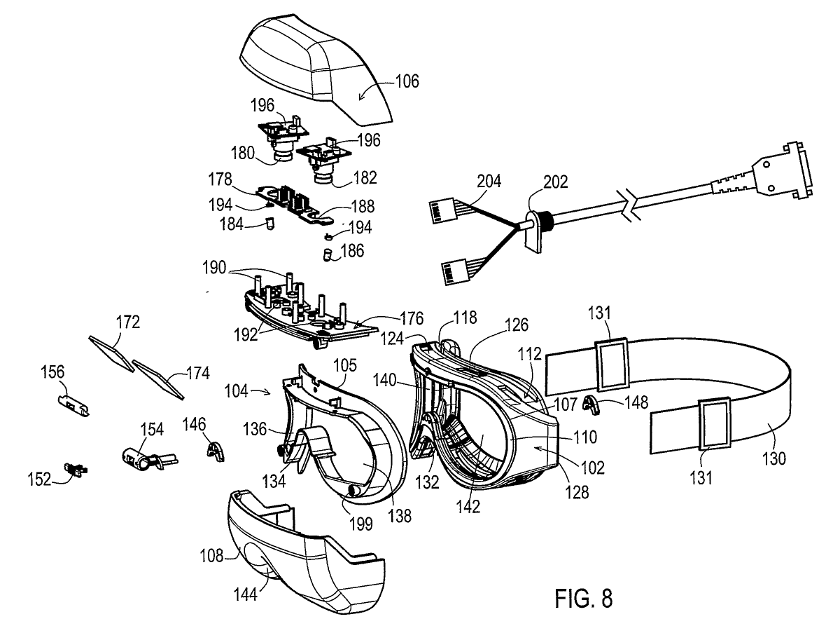

The assembly 150, shown in detail in the exploded view of FIG. 10 and the cross-sectional view of FIG. 9, includes a main bracket 154 that includes a horizontal shaft 166 and includes a front hole 164. The main bracket 154 also includes a rear brace portion 168 that includes shafts 170, at least one of which may have a threaded pin-insert. The shafts 170 pass through holes 145 in a front screw plate 146 that abuts the top of the nose indentation 134 of the frame 104. A rear screw plate 148 abuts the top of the nose indentation 132 on the facial side of the skirt 102. Screws or other fasteners then hold the screw plates 146, 148 to the frame 104 and skirt 102 to thereby hold the main bracket 154 in place.

To mount the infrared mirrors 172, 174 to the main bracket 154, a pole 156 is included. This pole 156 fits within the shaft 166 and is rotatable within the shaft. This pole 156 has flat portions 157 upon which the mirrors rest and are attached, such as by screws, adhesives, or other fasteners. Thus, the rotation of the pole 156 results in rotation of the mirrors 172, 174 relative to the frame 104 and skirt 102, and therefore, relative to the cameras 180, 182 and the eyes of the subject. This rotation allows for proper video capture of the eyes of the subject.

In order to provide the adjustment without handling the mirrors themselves, which could lead to smudges and damage, a button 152 having legs 158 and 160 is provided. The legs 158 and 160 pass through the hole 164 of the main bracket 154 and into the hole 162 of the pole 166. The hole 162 of the pole 166 is smaller than the hole 164 of the main bracket 154 such that the legs are maintained in a fixed position in the hole 162 of the pole 166 while remaining movable within the hole 164 of the main bracket 154. The size of the hole 164 may be chosen to constrain the amount of movement of the button 152, and hence the mirrors 172, 174 as can be seen in FIGS. 9 and 10. In the example shown, the hole 164 is positioned on the main bracket 154 such that when the button 152 is centered, the mirrors 172, 174 are at a 45 degree angle relative to the cameras 180, 182, and the size of the hole 164 permits a movement of +/−5 degrees from center.

In some instances, it may be desirable to completely block the vision of the subject and to present a dark field of view. To accomplish this, a cover 108 is provided that couples to the frame 104 and cabinet 106 as shown in FIG. 1B. In this example, the cover 108 includes a nose indentation 144. The cover 108 may be removably attached to the frame 104 in various ways. In the example shown, magnetic coupling is used by including magnets 198, 199 at various locations on the frame 104 and cabinet 106 while including magnetically attractive elements at matching locations within the cover 108, such as magnets having opposite polarity facing the frame 104 or having metal tabs.

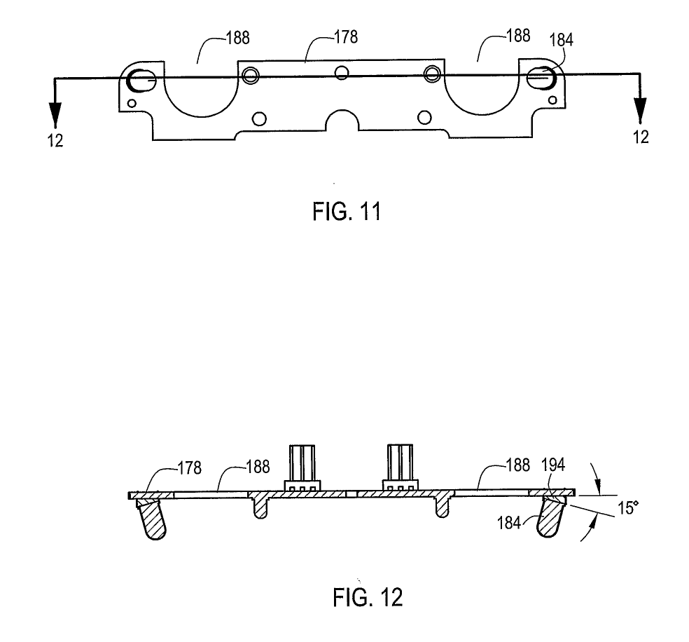

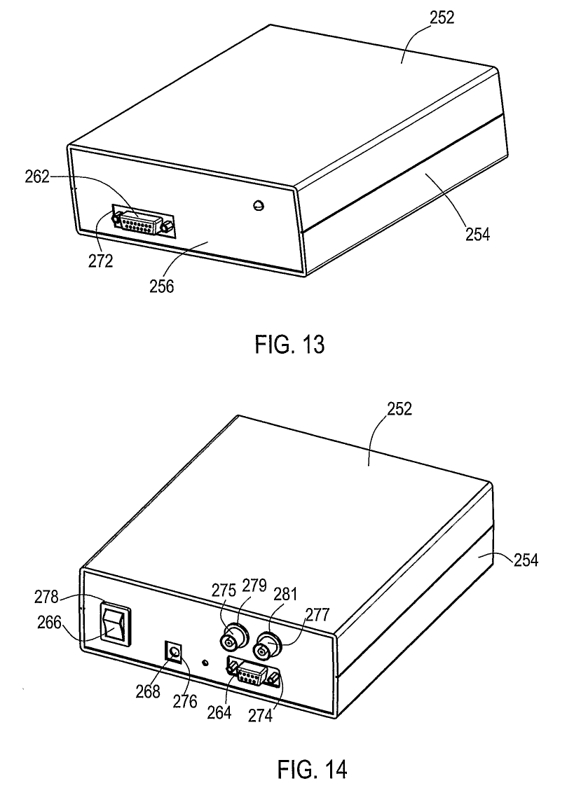

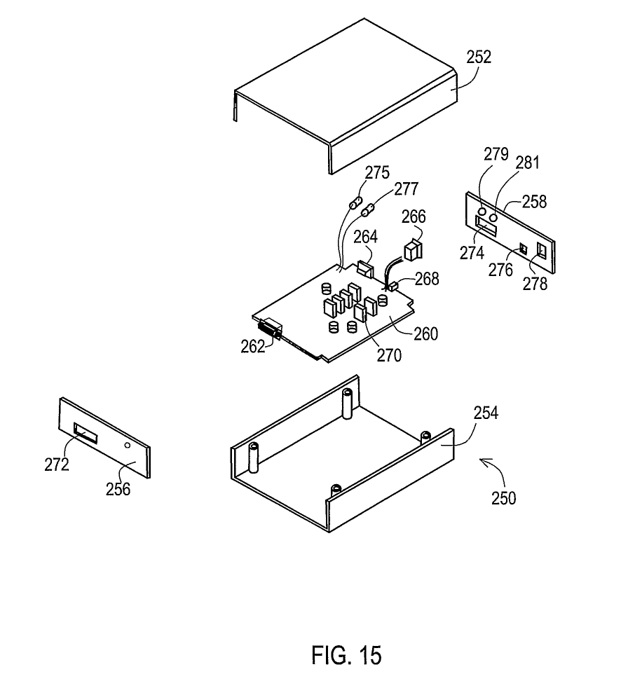

For embodiments where the goggles 100 include video capture equipment, a connection to a separate controller may be desired. Additional electronics needed to perform activation of the LEDs and cameras and to perform analysis, digitization, and/or enhancement to the video signals may be located in the controller rather than being included in the goggles 100 to prevent the goggles 100 from having a weight that is too great. FIGS. 13-15 show various views of one example of a control box 250.

The control box 250 includes a top portion 252 and bottom portion 254 that together form a housing. A front panel 256 and rear panel 258 are held in place by the top portion 252 and bottom portion 254.

A circuit board 260 having circuitry 270 located thereon is contained within the housing provided by the top portion 252 and bottom portion 254. The circuit board includes a first connector 262 that extends through a hole 272 in the front place 256. This connector 262 may attach via a cable to circuit board 178 of the goggles 100. As shown in FIGS. 2B and 8, the cabling 204 that is connected to the connector 262 and the circuit board 178 may pass through a grommet 202 connected to the cabinet 106. The circuit board 260 may also include a second connector 264 that extends through a hole 274 in the rear plate 258 to pass signals with another device such as a computer system. Additionally, conventional composite video connectors 275, 277 passing through holes 279, 281 may be present to output the video signals of the cameras to conventional video equipment such as video recorders or televisions. Additionally, the circuit board includes a DC power connector 268 that extends through hole 276 and a power switch 266 that extends through hole 278.

Thus, for embodiments of the goggles 100 that have video capabilities, those capabilities can be activated and accessed from external devices such as the control box 250. Video footage of the ocular movement may be obtained for various purposes, such as diagnosing vestibular disorders and the like.

While the invention has been particularly shown and described with reference to various embodiments thereof, it will be understood by those skilled in the art that various other changes in the form and details may be made therein without departing from the spirit and scope of the invention.

What is claimed is:

1. A pair of goggles, comprising:
an elastic skirt forming at least one aperture and having a curvature extending from a first side of the elastic skirt to a second side of the elastic skirt, the elastic skirt having a facial side, an outer rim, and a frame side, the outer rim defining a nose indentation, the outer rim including first, second and third damper portions partially extending into the elastic skirt, the first damper portion being formed along the curvature in a top portion of the outer rim opposite the nose indentation, the second damper portion being formed along the curvature in a bottom portion of the outer rim between the first side and the nose indentation, and the third damper portion being formed between the second side and the nose indentation; and
a frame attached to the frame side of the elastic skirt, the frame being more rigid than the elastic skirt, the frame having a curvature extending from a first side of the frame to a second side of the frame, the curvature of the frame coinciding with the curvature of the elastic skirt.

2. The pair of goggles of claim 1, wherein the frame defines a nose indentation that forms an aperture having a bay on each side of the nose indentation, the nose indentation of the frame coinciding with the nose indentation of the elastic skirt.

3. The pair of goggles of claim 2, wherein the frame comprises an upper chamber disposed above the two bays defined by the frame and opposite the nose indentation.

4. The pair of goggles of claim 3, further comprising a first infrared light source over and ahead of the first bay of the frame and a second infrared light source over and ahead of the second bay of the frame.

5. The pair of goggles of claim 4, further comprising:
a first infrared mirror disposed beneath the chamber and in alignment with the first bay of the frame;
a second infrared mirror disposed beneath the chamber and in alignment with the second bay of the frame; and
a mirror bracket affixed to the nose indentation of the frame, wherein the first infrared mirror has an inner side affixed to a first side of the mirror bracket and wherein the second infrared mirror has an inner side affixed to a second side of the mirror bracket.

6. The pair of goggles of claim 5, wherein the mirror bracket includes a first portion affixed to the first and second infrared mirrors and a second portion affixed to the nose indentation of the frame, wherein the first portion is rotatable relative to the second portion.

7. The pair of goggles of claim 6, further comprising a first infrared camera that is over and ahead of the first bay of the frame, that is focused on the first infrared mirror, and that is adjacent to the first infrared light source and a second infrared camera that is over and ahead of the second bay of the frame, that is focused on the second infrared mirror, and that is adjacent to the second infrared light source.

8. The pair of goggles of claim 7, wherein the first infrared light source is off-center relative to the first infrared mirror and is angled with respect to the frame so as to be aimed at the center of the first infrared mirror and wherein the second infrared light source is off-center relative to the second infrared mirror and is angled with respect to the frame so as to be aimed at the center of the second infrared mirror.

9. The pair of goggles of claim 1, further comprising a cover having a rigidity greater than the elastic skirt, the cover having an outer edge that mates to the frame.

10. The pair of goggles of claim 9, further comprising a magnetic coupling between the cover and the frame.

11. The pair of goggles of claim 1, wherein the elastic skirt comprises a thermoplastic elastomer.

12. A pair of goggles, comprising:
a skirt forming at least one aperture, the skirt having a facial side, an outer rim, and a frame side;
a frame attached to the frame side of the skirt, the frame being more rigid than the skirt, wherein the skirt forms an aperture having two bays separated by a narrow portion formed by a nose indentation of the skirt and wherein the frame forms an aperture having two bays aligned with the two bays of the skirt and separated by a narrow portion formed by a nose indentation of the frame that is aligned with the nose indentation of the skirt;
a first infrared mirror coupled to the frame and being disposed beneath the chamber and in alignment with the first bay of the frame;
a second infrared mirror coupled to the frame and being disposed beneath the chamber and in alignment with the second bay of the frame;
a first infrared light source coupled to the frame such that the first infrared light source is over and ahead of the first bay of the frame, the first infrared light source being off-center relative to the first infrared mirror and being angled with respect to the frame so as to be aimed toward the center of the first infrared mirror; and
a second infrared light source coupled the frame such that the second infrared light source is over and ahead of the second bay of the frame, the second infrared light source being off-center relative to the second infrared mirror and being angled with respect to the frame so as to be aimed toward the center of the second infrared mirror.

13. The pair of goggles of claim 12, wherein the skirt is elastic and wherein the outer rim of the elastic skirt includes damper portions partially extending into the elastic skirt.

14. The pair of goggles of claim 12, wherein the skirt has a curvature extending from a first side of the skirt to a second side of the skirt and wherein the frame has a curvature extending from a first side of the frame to a second side of the frame, the curvature of the frame coinciding with the curvature of the elastic skirt.

15. The pair of goggles of claim 14, further comprising a mirror bracket that couples the first and second infrared mirrors to the frame, wherein the mirror bracket includes a first portion affixed to the first and second infrared mirrors and a second portion affixed to the nose indentation of the frame, wherein the first portion is rotatable relative to the second portion.

16. The pair of goggles of claim 14, further comprising a first infrared camera that is over and ahead of the first bay of the frame, that is focused on the first infrared mirror, and that is adjacent to the first infrared light source, and a second infrared camera that is over and ahead of the second bay of the frame, that is focused on the second infrared mirror, and that is adjacent to the second infrared light source.

17. The pair of goggles of claim 12, further comprising a cover having a rigidity greater than the skirt, the cover having an outer edge that mates to the frame.

18. The pair of goggles of claim 17, further comprising a magnetic coupling between the cover and the frame.

19. A pair of goggles, comprising:
a skirt forming at least one aperture, the skirt having a facial side, an outer rim, and a frame side;
a frame attached to the frame side of the skirt, the frame being more rigid than the skirt, wherein the skirt forms an aperture having two bays separated by a narrow portion formed by a nose indentation of the skirt and wherein the frame forms an aperture having two bays aligned with the two bays of the skirt and separated by a narrow portion formed by a nose indentation of the frame that is aligned with the nose indentation of the skirt;
a first infrared mirror in alignment with the first bay of the frame;
a second infrared mirror in alignment with the second bay of the frame; and
a mirror bracket having a first portion affixed to the nose indentation of the frame and having a second portion affixed to the first and second infrared mirrors, wherein the second portion is retained by and is movable relative to the first portion.

20. The pair of goggles of claim 19, wherein the mirror bracket is substantially transparent.
